(19) United States
(12) Patent Application Publication
(10) Pub. No.: US 2023/0166214 A1
(43) Pub. Date: Jun. 1, 2023

(54) AIR PURIFICATION AND AIR CONDITIONING SYSTEM

(71) Applicant: Clean Air Zone Inc., Corona, NY (US)

(72) Inventors: Serge B. HOYDA, Great Neck, NY (US); Gregg Russell DICKINSON, Briarwood, NY (US)

(73) Assignee: Clean Air Zone Inc., Corona, NY (US)

(21) Appl. No.: 18/070,466

(22) Filed: Nov. 29, 2022

Related U.S. Application Data
(60) Provisional application No. 63/283,715, filed on Nov. 29, 2021.

(51) Int. Cl. B01D 53/84; A61L 9/14; B01D 53/34

(52) U.S. Cl. CPC B01D 53/84; A61L 9/145; B01D 53/346; A61L 2209/111; A61L 2209/16; A61L 2209/134

(57) ABSTRACT

There is an air purification system comprising, at least one housing, at least one pump configured to pump an enhanced fluid through the housing. at least one fan configured to draw air into the housing. With this embodiment, the air flow of the air is being cleaned and flows in a direction transverse to the direction of flow of the enhanced fluid. There can be at least one sensor configured to determine a level of biological impurities in the air. There can also be at least one controller configured to control a rate of movement of said at least one fan based upon a level of impurities detected by said at least one sensor.

### CROSS REFERENCE TO RELATED APPLICATIONS

This application is a non-provisional application that claims priority from U.S. Provisional Application Ser. No. 63/283,715 filed on Nov. 29, 2021, the disclosure of which is hereby incorporated by reference.

### BACKGROUND OF THE INVENTION

There is disclosed an air purification that can be incorporated in with an air conditioning system. The air purification system can be formed either separately from the air conditioning system or integrated with the air conditioning system. With the rise of respiratory ailments as well as environmental toxins, there is a need to create a clean air environment which allows for integration with air conditioning systems.

For the purpose of clearly defining the terms associated with this application, below are the defined terms. An indoor facility can include but is not limited to a house, home, apartment building, commercial, industrial, retail stores, or centers, offices, hotels, hospitals, nursing homes, schools and/or universities or any other type of indoor facilities. These indoor facilities' rooms are also known as rooms. A natural biological purification solution will hereby be known as a bio solution. A biological or bio solution in combination with water will be known as an enhanced fluid. An air conditioning system will be known as a HVAC system or vice versa. An air purification system is a system which uses the bio solution or any other suitable air purifying system such as HEPA and/or ultraviolet light. Negative pressure inside of a room is created by fans drawing air into a room and through the room. Air borne contaminants shall include but are not limited to air borne particles that are equal to or more than 0.0001μ, allergens such as pollen, bacteria, odors, viruses, germs, smoke such as cigarette smoke, chemical fumes, and other pollutants such as bacteria, mold and ozone. This bio solution is substantially or 100% natural which can exist through low energy consumption and works well with HVAC. The types of particles that this has been tested with include but are not limited to corona virus, wood smoke, mold spores, legionella, H1N1, Clostridium difficile, Candida albicans, Mycobacterium tuberculosis, influenza, B/Lee/40, gas remediation, reduction of carbon monoxide, reduction of diesel combustion gasses PM 2.5 particulate &VOC emission, bacteria.

### SUMMARY OF THE INVENTION

There is an air purification system comprising, at least one housing, at least one pump configured to pump an enhanced fluid through the housing. at least one fan configured to draw air into the housing. With this embodiment, the air flow of the air is being cleaned and flows in a direction transverse to the direction of flow of the enhanced fluid. There can be at least one sensor configured to determine a level of biological impurities in the air. There can also be at least one controller configured to control a rate of movement of said at least one fan based upon a level of impurities detected by said at least one sensor. The system can be configured to monitor and control a pump, a fan, a hi/lo sensor and a solenoid so that the system operates to effectively and efficiently remove impurities in the air.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a side view of a home having an air conditioning system;

FIG. 2 is a view of a computer system for controlling an air conditioning system;

FIG. 3 is a side view of an air purification system which can be used in series with an air conditioning system;

FIG. 4A is a view of a perforated wall or tray that can be used with the air purification system;

FIG. 4B is a perspective view of the perforated tray;

FIG. 5A is a perspective view of at least one hole that is formed as a module in the perforated tray;

FIG. 5B is a side view of the hole that is formed in the perforated tray;

FIG. 6 is an end view of the hole of the perforated tray;

FIG. 7 is a plan view of the air purification system that can be used together with the air conditioning system;

FIG. 8 is an exploded view of the air purification system that can be used with the air conditioning system;

FIG. 9 is a top view of the return for the biological fluid for the air purification system;

FIG. 10A is a side cross-sectional view of the air purification system for use with an air conditioning system;

FIG. 11 is a side view of another air purification system for use with an air conditioning system or for use with air treatment of clean rooms or grow rooms;

FIG. 12 is a side perspective view of the system shown in FIG. 11;

FIG. 13 is an exploded view of the air purification system shown in FIG. 12;

FIG. 14 is a view of a portion of the air purification system shown in FIG. 13;

FIG. 15 is a view of another air purification system for use with air conditioning or air treatment of hot house or hydroponic systems;

FIG. 16 is a side cut away vie of the air purification system of FIG. 15;

FIG. 17 is a perspective view of the air purification system of FIG. 15;

FIG. 18 is a perspective view of a plate for use with the air purification system of FIG. 15; and

FIG. 19 is a flow chart for the processor shown in FIG. 2 operating the air purification system.

### DETAILED DESCRIPTION OF PREFERRED EMBODIMENTS

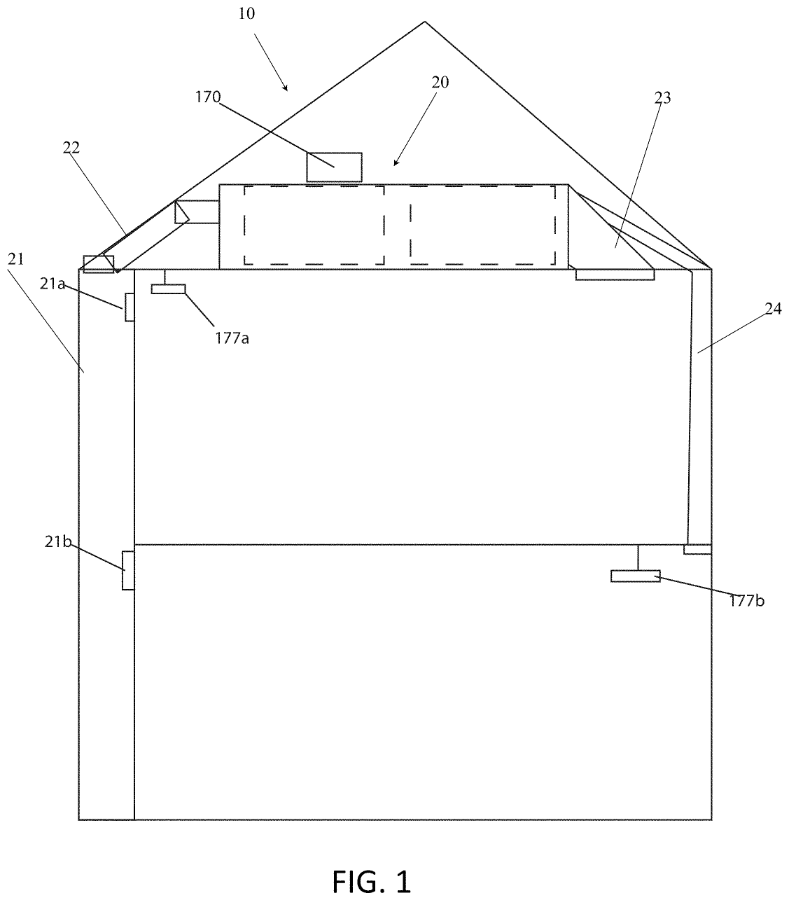

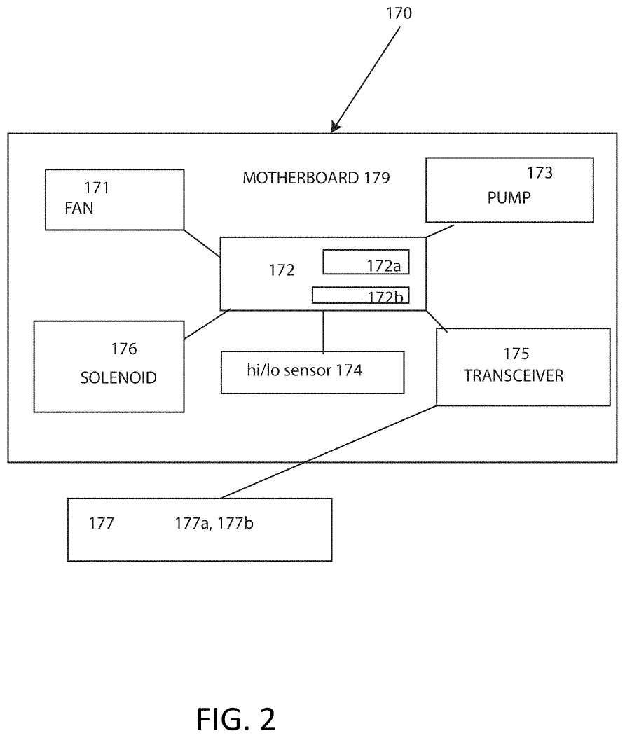

FIG. 1 is a side view of a home having an air conditioning system which discloses a house 10 having an attic which houses an air purification system which is in series with an air conditioning system and which is in communication with the air in the house via channels 21, 22, 23, and 24. Channels 21 and 22 feed air into the house through openings such as opening or vent 21a or opening or vent 21b while channels 23 and 24 form separate returns. Therefore, the system acts as a close ended system which recycles air throughout the house which can be a residence or building a such that with each passing of the air, the quality of the air consistently improves. This system includes a controller 170 shown in FIG. 2 as well as sensors 177a and 177b positioned throughout the house. The system is designed so that it can be positioned as a single unit 40 comprising both an air purification system and an air conditioning system or as separate systems comprising a separate air purification system 50 and separate air conditioning system 60 coupled together in series.

FIG. 2 is a schematic block diagram of the controller system 170. The controller system 170 comprises a central controller 172 which includes a microprocessor 172a, and an onboard memory 172b. The microprocessor 172a and memory 172b are configured to control the fan 171, as well as a pump 173. There is also a hi/lo sensor 174 which determines the level of fluid in the system, a transceiver 175 as well as a solenoid 176 which is configured to be selectively opened or closed to allow water flow into the housing. The controller 172 is configured to communicate with outside sensor(s) 177 including sensors 177a and 177b shown in FIG. 1.

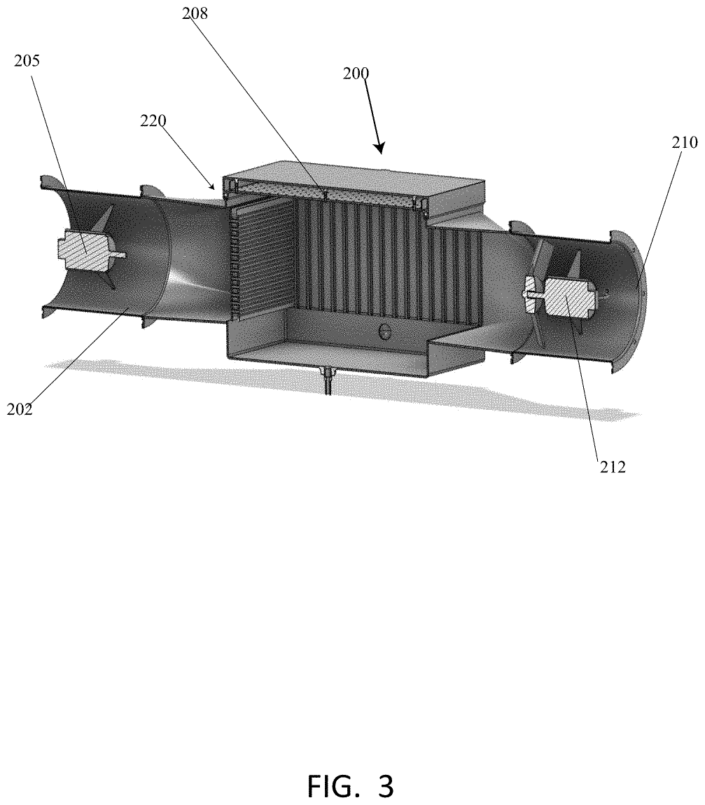

FIG. 3 is a side view of an air purification system which can be used in series with an air conditioning system. With this view there is a purification section which is in communication with the air channels 21, 22, 23, and 24. This system 200 includes fans 205 and 212 which feeds air through channels 202 and 210. In at least one embodiment, channel 202 connects to channel 23, while channel 210 connects to channel 22. There is a chamber 208 which sits between channels 202 and 210. There are also fans 205 and 212 which are configured to keep the air flowing through the system.

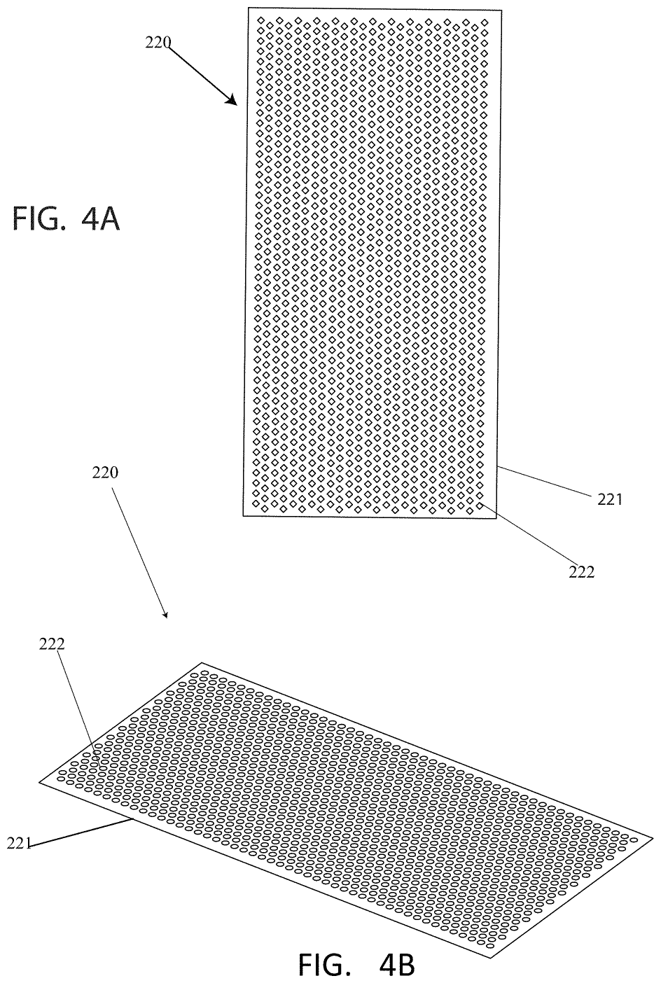

FIG. 4A is a view of a perforated wall or tray that can be used with the air purification system. The perforated wall 220 is configured to sit across the airflow inside of the chamber. This perforated wall 220 includes a plurality of holes 222 positioned therein, and a frame 221 positioned around the outside perimeter. FIG. 4B is a perspective view of the perforated tray showing the plurality of holes 222 and the frame 221.

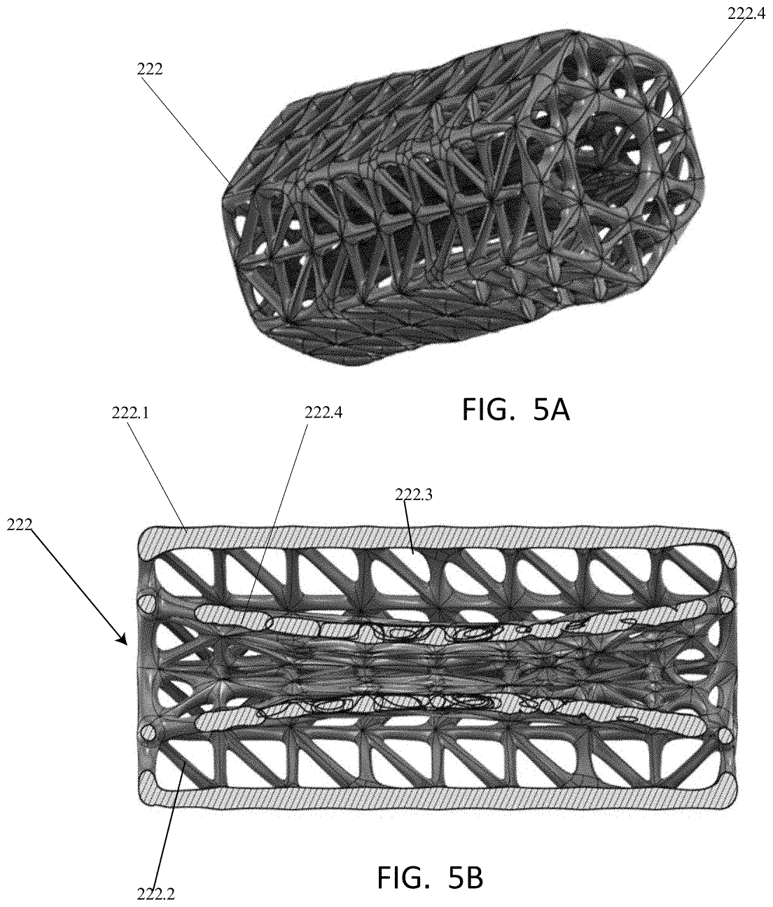

FIG. 5A is a perspective view of at least one hole that is formed as a module in the perforated tray. The holes can be formed as a form of honeycomb patterned cells 222. There is a center hole 222.4 as well as struts formed around the honeycomb pattern.

For example, FIG. 5B is a side view of the hole that is formed in the perforated tray, wherein there is shown central hole 222.4, outer sheath 222.1, struts 222.2, and holes 222.3. The honeycomb patterned hole is configured to accept biological material flowing down from gravitational force while air moves across this honeycomb pattern through hole 222.4 and through holes 222.3 so that there is increased air to biological material interaction. With this design, biological material is positioned above this perforated tray, and then is allowed to cascade or flow down across this tray so that it interacts with air flowing in a transverse manner through hole 222.4 from one end and out the other side.

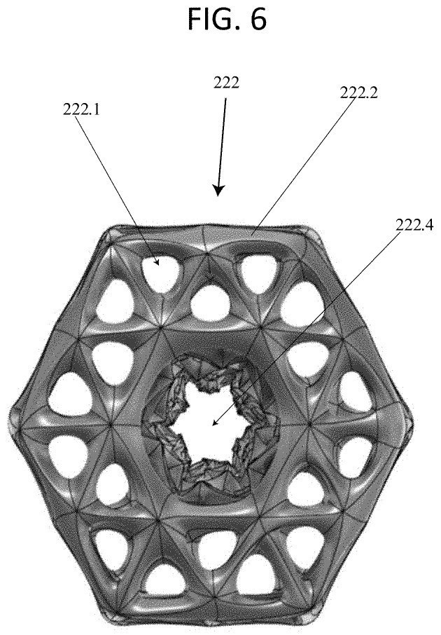

FIG. 6 is an end view of the hole of the perforated tray, this view shows central hole 222.4, as well as holes 222.2 and struts 222.1. The honeycomb pattern 222 is configured to provide for consistent flow of fluid across the struts of the honeycomb pattern thereby increasing interaction of air with the biological material.

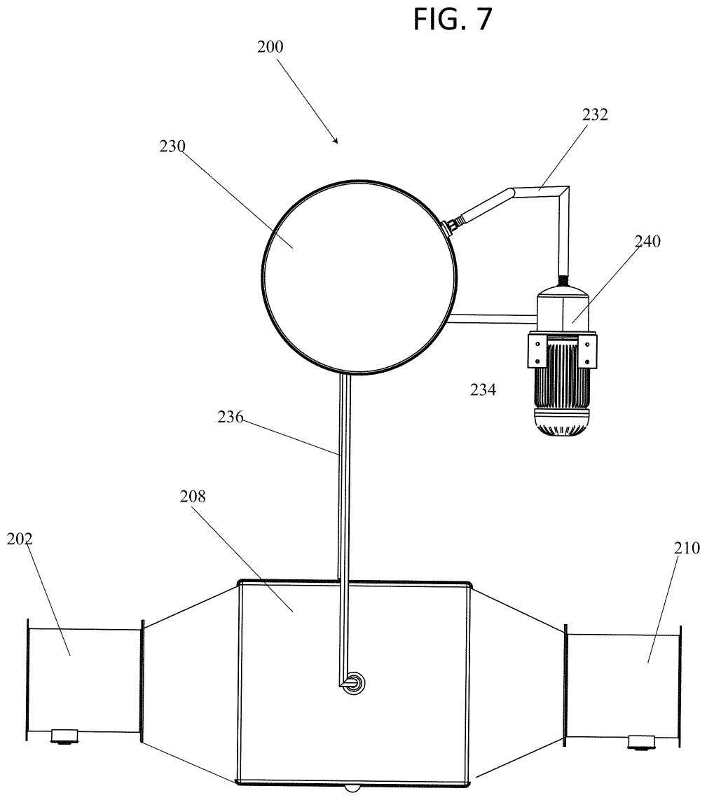

FIG. 7 is a plan view of the air purification system that can be used together with the air conditioning system. In this view, there is the main biological air flow system 200 which has a first channel 202 and an oppositely spaced channel 210. There is a central channel or chamber 208 positioned between these two channels 202 and 210. A tank 230 is configured to feed biological material via tube 236 into central channel or chamber 208 to allow fluid to flow across each of the perforated trays 220. There is a pump 240 which is configured to allow fluid to flow from chamber 230 via channel 234 into pump 240, and then out through channel 232 so that the biological material that is stored inside of chamber 230 is circulated via pump 240 and then pumped out of the chamber 230 and into the central channel or chamber 208.

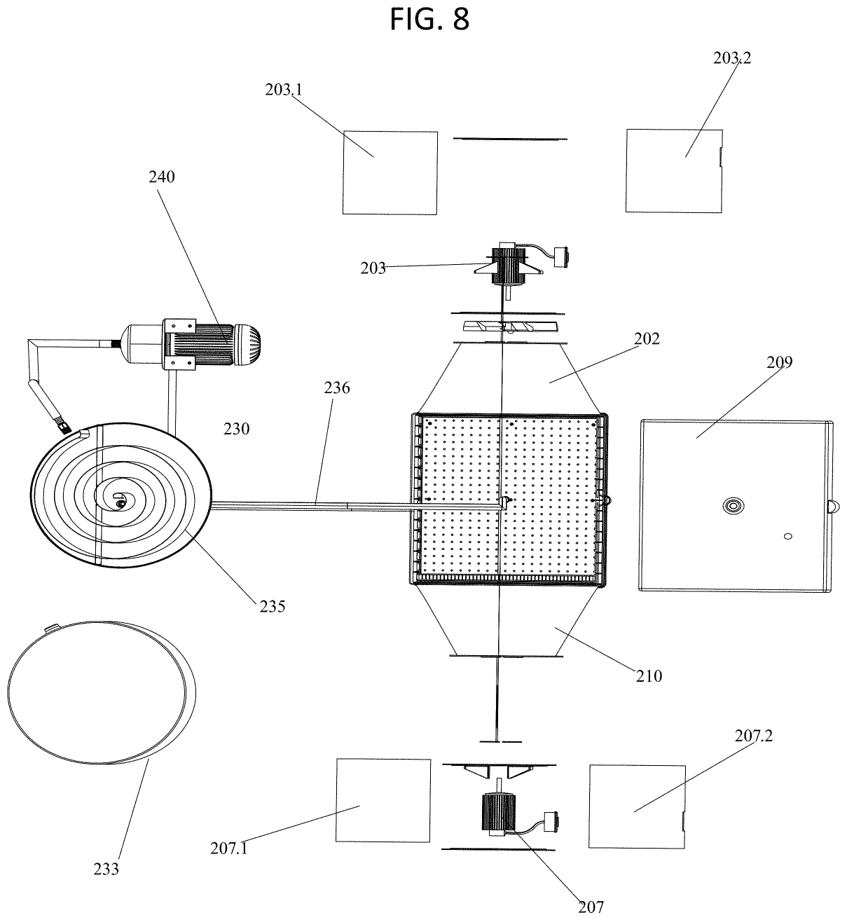

FIG. 8 is an exploded view of the air purification system that can be used with the air conditioning system. With this view there is a view of the top plates 203.1 and 203.removed. In addition, body or chamber 208 shows a perforated screen as well. A top 209 is shown removed from the body section or chamber 208 which shows a bottom screen which is configured to allow fluid to flow therethrough and collect at a bottom reservoir. Thus is shown, there is disclosed an auxiliary tank 230 which has a central spiral 235 which is in the form of an ever increasing corkscrew which allows fluid from the central region to spiral outward, or depending on flow, spiral inward, depending on the flow of fluid through the system. As shown with the panels removed 203.1 and 203.1 there is a first fan 203, disposed inside of chamber 202, a central chamber 208 has a tray which is a perforated tray, and which as shown as by removed top plate 209. Inside the body 208 is a bottom tray that is perforated which then feeds into chamber 230 allowing for circulation of the biological fluid between chamber 230 and chamber 208 via line 236. Assisting in this circulation is pump 240 which provides pressure via line 232 into chamber 230 and which drives the circulation of the biological fluid between chamber 230 and chamber 208. Chamber 230 is exposed for having spiral 235 with the removal of top 233.

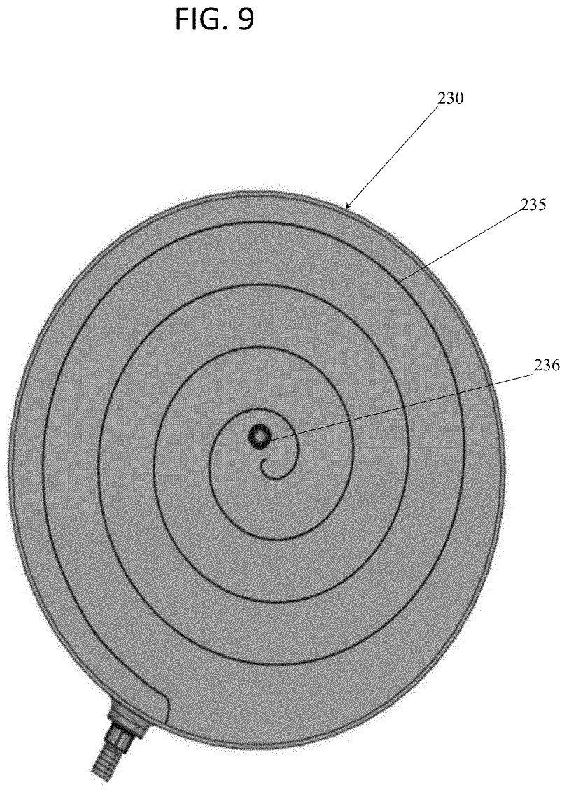

FIG. 9 is a top view of the return for the biological fluid for the air purification system showing the spiral 235 and the line connection 236.

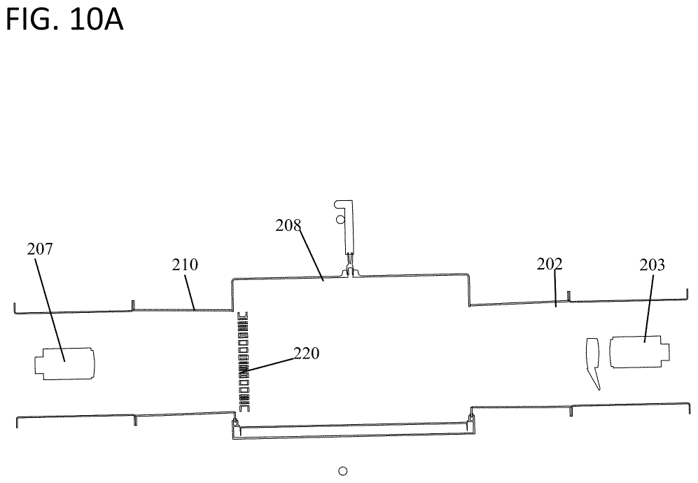

FIG. 10A is a side cross-sectional view of the air purification system for use with an air conditioning system, this view shows chambers 202, and 210 with fans 207 and 203 disposed therein. There is also shown chamber 208 as well as side cross-sectional view of tray 220. Tray 220 is configured to allow biological fluid to flow down from a top position to a bottom position while having air passing through in a transverse manner thereby creating an interaction between the air and the biological fluid.

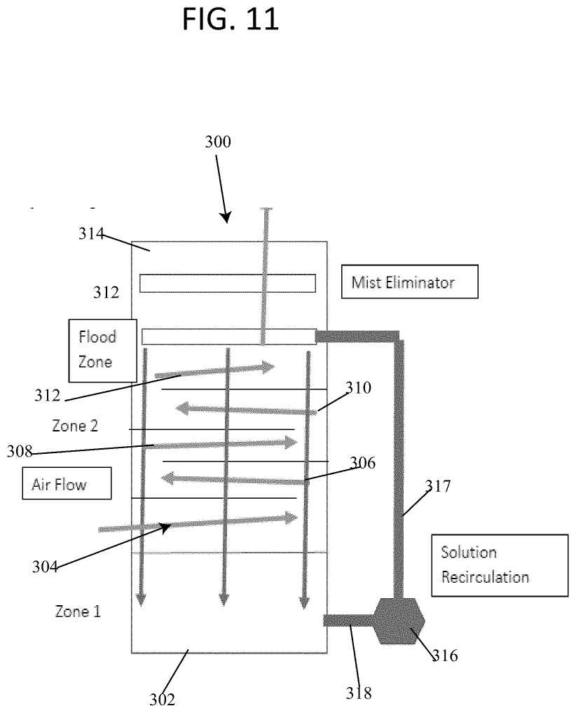

FIG. 11 is a side view of another air purification system for use with an air conditioning system or for use with air treatment of clean rooms or grow rooms. In this view there is a self-contained system 300 which has biological fluid flow from a top position to a bottom position, with air flowing up the container in an opposite direct as shown by the arrows. Thus, the flow of water in the opposite direction to the flow of air creates an interaction between the biological solution contained in the water and the air, thereby allowing for the removal of biological material from the air. Thus, the top position 314 allows for the deposition of biological fluid, while the different levels 304, 306, 308, 310 and 312 of trays allows for the constant flow of air in an opposite direction to the flow of biological fluid creates a cleaning of the air and purification of the air in the closed chamber 302. A pump 316 circulates this fluid through lines 318 and 317 which allows for continuous circulation of this fluid throughout the system.

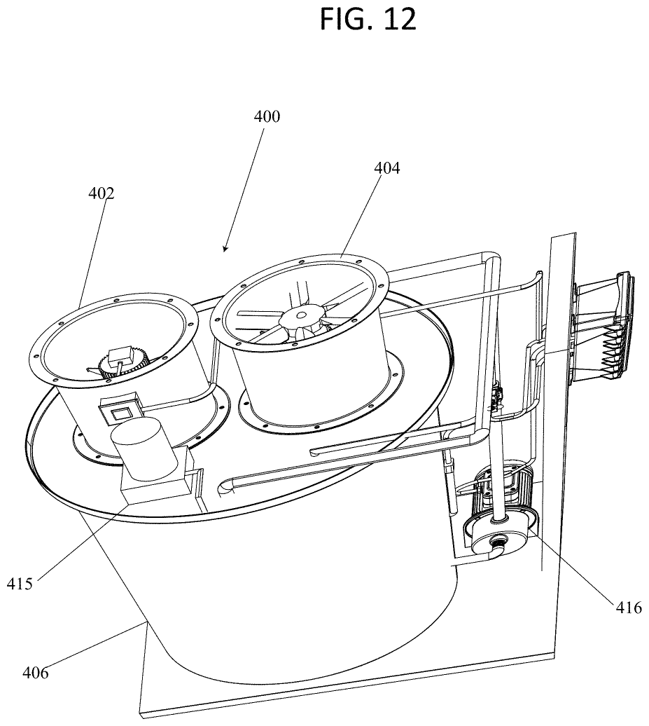

FIG. 12 is a side perspective view of the system shown in FIG. 11, this is a slightly modified version which shows fan 404 which creates a negative pressure within chamber 406. There is also a mixer 402 which is in communication with a pump 416 configured to pump biological material from a bottom position on the chamber 406 to a top position.

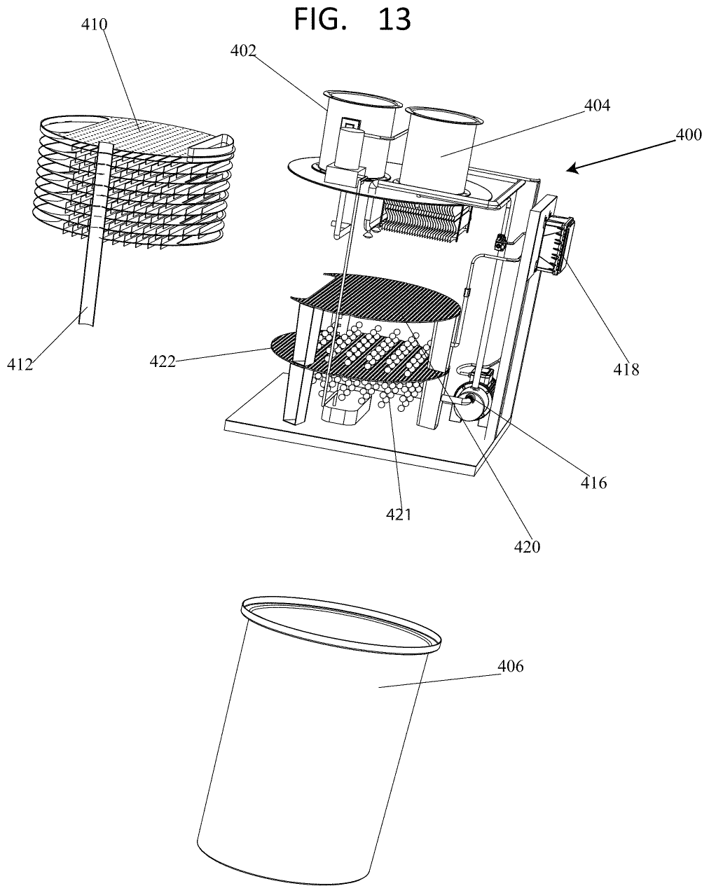

FIG. 13 is an exploded view of the air purification system shown in FIG. 12. With this view there is shown a chamber 406 along with a series of trays 410. These trays comprise slanted trays allowing for fluid flow down the trays. Each level of these trays also comprises perforated plates allowing fluid flow down the trays. A channel 412 allows for fluid flow from a bottom position to a top position within the trays. As shown pump 416 pumps the biological fluid from a bottom position to a top position. Fan 404 creates a negative pressure within the chamber 406 while a controller 418, controls both the fan 404 and the pump 416. There are also two different settling levels for the biological fluid at a first level 420 and a second level 422. Disposed between levels 420 and 422 are balls 421, which allow for the mixing of the biological material once it has flowed down from a top position to a bottom position within the chamber.

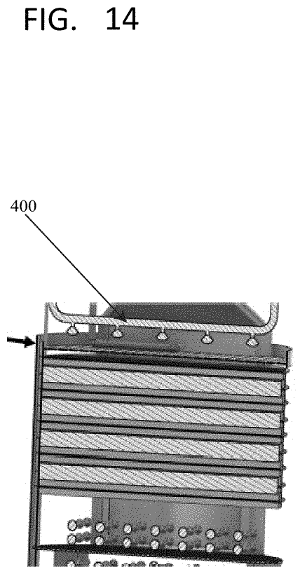

FIG. 14 is a view of a portion of the air purification system shown in FIG. 13 which shows the different trays shown in a slanted orientation allowing for the fluid to flow from a top position to a bottom position while the negative pressure of air within the system flows from a bottom position to a top position, thereby creating an interaction between the air and the fluid.

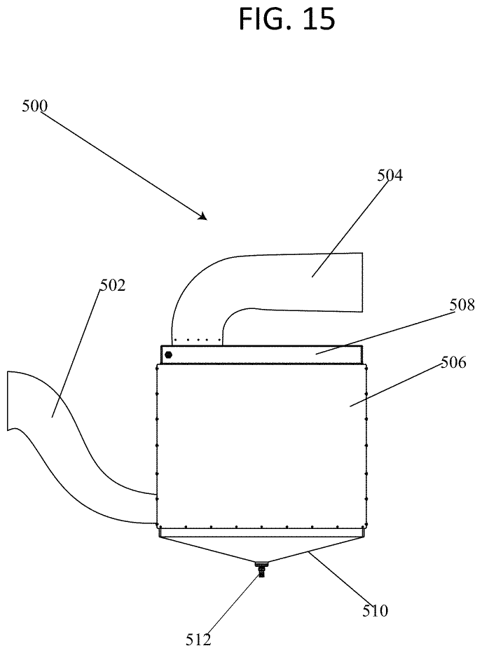

FIG. 15 is a view of another air purification system for use with air conditioning or air treatment of hot house or hydroponic systems. This system 500 includes a chamber 506, an air intake 502, as well as an air outflow 504. A top tray section 508 is for allowing fluid to collect and then flow from a top position inside chamber 506 to a bottom position in a collecting tray 510. An outflow spout 512 is positioned inside of the chamber 506.

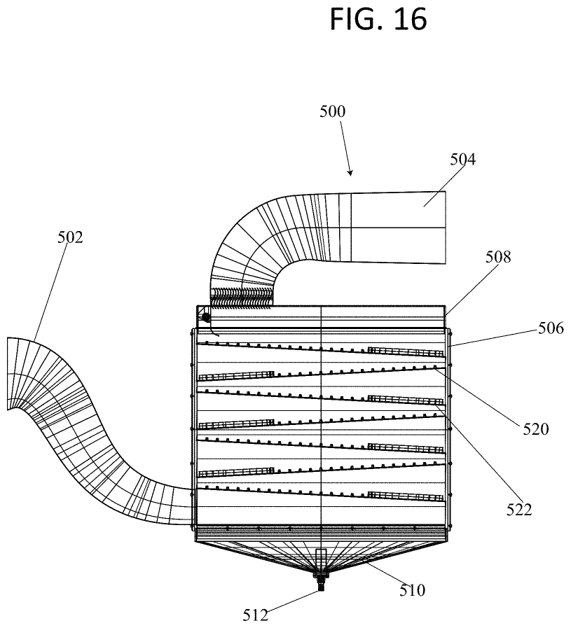

FIG. 16 is a side cut away vie of the air purification system of FIG. 15 which shows intake 502, and air outflow chamber 504. There is a chamber 506 which has a plurality of trays 520 wherein each tray is made from a perforated plate having a hole 522. Fluid is fed into a top tray 508 and flows from this top tray 508 down across trays 520, through associated perforations while holes 522 are configured to allow air to flow opposite the flow of water, wherein the air flows from a bottom position as fed by intake or chamber 502 and up and out through chamber 504.

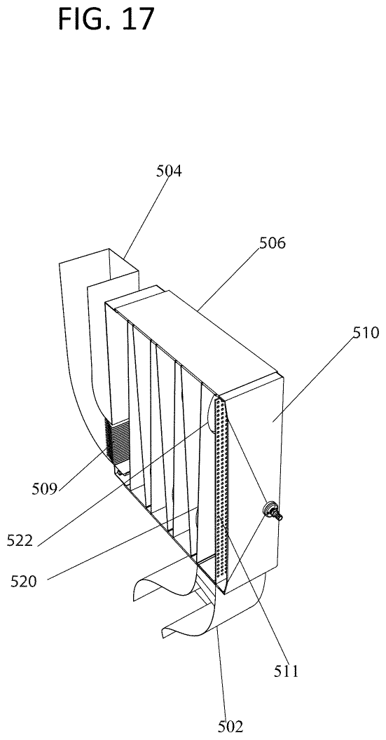

FIG. 17 is a perspective view of the air purification system of FIG. 15 which shows this system having intake 502, outflow chamber 504 wherein there is shown perforated tray 509 at a top position as well as perforated tray 511 shown at a bottom position of chamber 506.

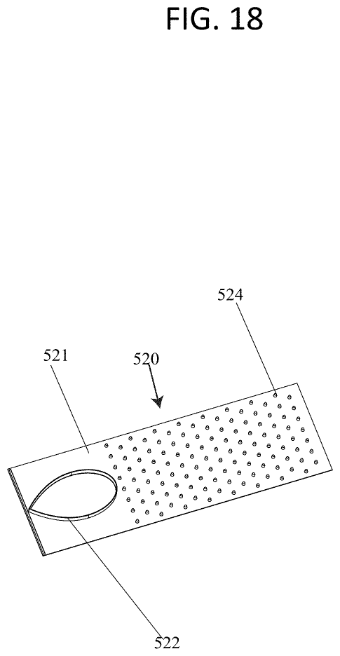

FIG. 18 is a perspective view of a plate for use with the air purification system of FIG. 15. This view shows tray 520 having perforations 524 as well as air flowing hole 522 to allow for air to flow from a bottom position to a top position. A portion 521 of tray 520 is a solid portion of this tray.

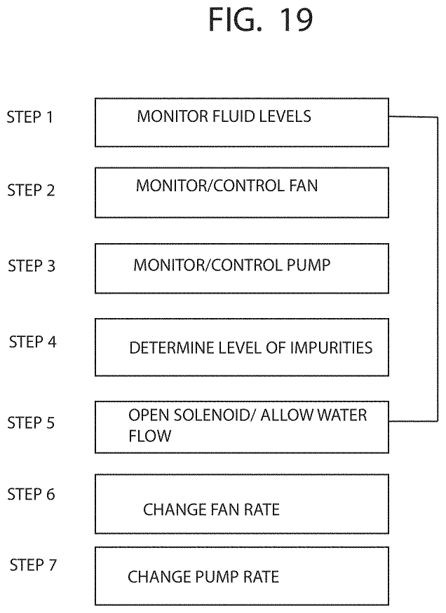

FIG. 19 is a view of the flow chart for the operation of the processor 172 comprising the microprocessor 172a and the memory 172b in controlling the peripheral components such as any one of the fan 171, the pump 173, the hi/lo sensor 174, the transceiver 175, the solenoid 176 and the air purification sensors 177 including a first sensor 177a and a second sensor 177b.

For example, the process starts in step 1 wherein the processor 172 monitors fluid levels in the system. The monitoring of the fluid levels is through the readings from the hi/lo sensor 174 feeding the fluid levels into the processor 172 such as microprocessor 172a. Microprocessor 172a reads the pre-determined levels that would be sufficient to effectively operate the device. If the fluid levels are too low the system proceeds to step 5 wherein the system opens the solenoid such as solenoid 176 to allow more fluid into the system such as water. Next, in step 2 the system including processor 172 monitors the fan rate by monitoring the rpm's of the fan 171. Next, in step 3 the system monitors the level of the circulating pump 173 by monitoring the fluid flow/and/or energy use in the pump. Next, in step 4 the system including processor 172 determines the level of impurities by reading the impurity levels from sensor(s) 177 including sensor 177a and 177b. If the system determines that the impurity levels are too high the system can selectively move to step 5 to open the solenoid to allow water into the system, or in step 6 change the fan rate of fan 171 or change the pump rate of pump 173 to selectively reduce the impurity levels in the air because increasing the fan rate would increase the air flow while increasing the pump rate would increase the enhanced fluid flow thereby creating increased interactions between air and enhanced fluid in the system. When the impurity levels are reduced, then the system would proceed back to steps 6 and 7 to selectively reduce the fan rate and/or the pump rate.

Thus, there is created a dynamic air cleansing system for biological rooms such as greenhouses which can clean either in a continuous process or in a multi stage process.

Accordingly, while at least one embodiment of the present invention have been shown and described, it is to be understood that many changes and modifications may be made thereunto without departing from the spirit and scope of the invention as defined in the appended claims.

What is claimed is:

1. An air purification system comprising:
at least one housing;
at least one tray disposed in said at least one housing;
at least one pump configured to pump an enhanced fluid through the housing;
at least one level sensor configured to determine a level of the enhanced fluid through the housing;
at least one fan configured to move air through the housing; and
wherein the enhanced fluid is configured to flow through the housing in a direction substantially transverse to the flow of the enhanced fluid.

2. The air purification system as in claim 1, wherein said at least one tray is a perforated tray.

3. The air purification system as in claim 1, wherein said at least one tray is configured to pump the enhanced fluid from a first level to a higher level.

4. The air purification system as in claim 1, wherein said at least one tray comprises a plurality of cells formed in a honeycomb shape.

5. The air purification system as in claim 1 wherein the air purification system is positioned adjacent to an air conditioning system.

6. The air purification system as in claim 1, wherein the air purification system is integrated with an air conditioning system.

7. The air purification system as in claim 1, wherein said at least one tray comprises a plurality of trays stacked inside of the housing.

8. The air purification system as in claim 7, wherein said plurality of trays comprises a plurality of substantially horizontally extending trays spaced apart from each other.

9. The air purification system as in claim 1, wherein said at least one tray comprises a tray having a perforated section and at least one air flow section.

10. The air purification system as in claim 9, wherein said air flow section comprises at least one hole having a rim around the hole to deflect a flow of enhanced fluid in the housing.

11. The air purification system as in claim 10, wherein said at least one tray comprises a plurality of trays, and wherein each of said plurality of trays are positioned in said housing in a slanted manner with at least one end being higher than an opposite end inside of the housing.

12. The air purification system as in claim 1, further comprising at least one microprocessor and at least one sensor, wherein said at least one sensor is configured to determine a level of impurities in the air and wherein said microprocessor is configured to change a setting of said at least one pump and said at least one fan depending on a reading of the level of impurities in the air.

13. The air purification system as in claim 1, further comprising at least one enhanced fluid chamber which is separate from said at least one housing.

14. The air purification system as in claim 1, wherein said enhanced fluid chamber comprises at least one spiral separator for guiding the enhanced fluid inside of the chamber.

15. The air purification system as in claim 1, wherein said housing has an air intake positioned at a lower region of the housing and an air outflow positioned at an upper region of the housing.

16. The air purification system as in claim 1, further comprising at least one sprayer positioned inside of the housing wherein said at least one sprayer is configured to spray the enhanced fluid inside of the housing.

17. The air purification system as in claim 16, wherein said at least one sprayer comprises a spray manifold comprising a plurality of sprayers inside of said housing.

18. The air purification system as in claim 1, wherein said at least one tray is oriented substantially vertically inside of said housing.

19. The air purification system as in claim 1, further comprising a plurality of movable objects inside of the housing, wherein said movable objects are configured to mix the enhanced fluid inside of the housing.

20. The air purification system as in claim 19. wherein said movable objects comprise balls which are configured to move and mix the enhanced solution inside of the housing.
